(19) United States
(12) Patent Application Publication
(10) Pub. No.: US 2010/0081688 A1
(43) Pub. Date: Apr. 1, 2010

(54) Process for the preparation of montelukast, and intermediates therefor

(75) Inventor: Srinivas Laxminarayan Pathi, Karnataka (IN)

Correspondence Address: CONLEY ROSE, P.C., 5601 GRANITE PARKWAY, SUITE 750, PLANO, TX, 75024, US

(73) Assignee: CIPLA LIMITED, Mumbai (IN)

(21) Appl. No.: 12/441,129

(22) PCT Filed: Sep. 14, 2007
(86) PCT No.: PCT/GB07/03510

§ 371 Date: Mar. 12, 2009

(30) Foreign Application Priority Data
Sep. 15, 2006 (IN) 1476/MUM/2006

(51) Int. Cl. A61K 31/47; C07D 215/18

(52) U.S. Cl. 514/311; 546/180

(57) ABSTRACT

The invention relates to processes for making montelukast and to intermediates for use in the process, in particular compounds of formulas 2-7: L=OAc, OTs, OTf5OMs; where X=Cl, Br, I.

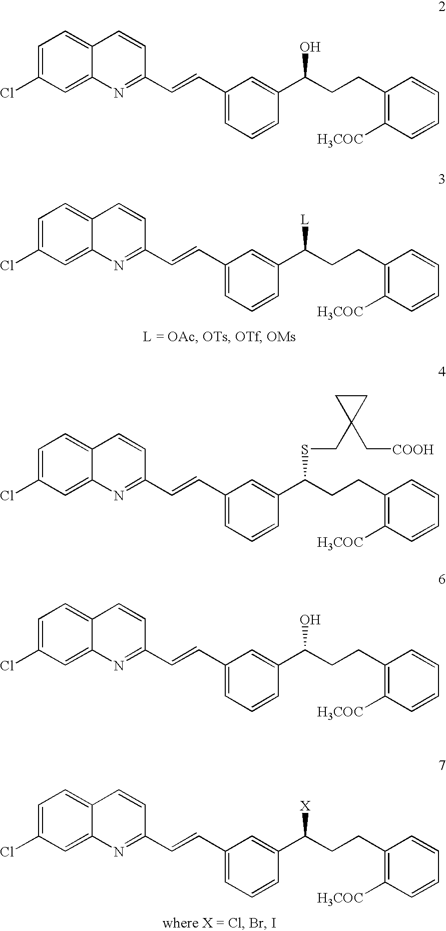

### TECHNICAL FIELD

The present invention relates to an improved process for the preparation of montelukast and its salts via novel intermediates. The invention also relates to said intermediates, per se.

### BACKGROUND AND PRIOR ART

Montelukast is a selective, reversible leukotriene receptor antagonist and chemically known as 1-[[[(1R)-(3(2-(7-Chloro-2-quinolinyl)pethenyl]phenyl}-3-[2-(1-hydroxy-1-methylethyl)phenyl]propyl]thio]methyl]cyclopropaneacetic acid represented by the Formula I.

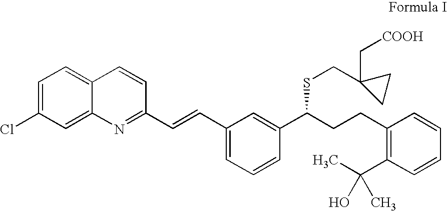

Leukotrienes were first discovered in the 1930's as potent mediators of inflammation and given the name slow-reacting substance of anaphylaxis. Montelukast monosodium salt (montelukast sodium) is commonly used for treatment of asthma.

U.S. Pat. No. 5,565,473 (EPO480717) discloses a genus of pharmaceutically useful compounds that encompasses montelukast and salts thereof. Example 161 in connection with example 146 of U.S. Pat. No. 5,565,473 discloses the synthesis of montelukast sodium which includes reacting 2-(2-(2-(3(S)-(3-(2-(7-chloro-2-quinolinyl)-ethenyl)phenyl)-3-(methanesulfonyloxy) propyl)phenyl)-2-propoxy) tetrahydropyran with methyl1-(acetylthiomethyl)cyclopropane acetate in presence of hydrazine, cesium carbonate in acetonitrile as solvent to get the methyl ester of montelukast in pyran protected form. The protected compound is further reacted with pyridinium p-toluene sulfonate, sodium hydroxide in a mixture of methanol and tetrahydrofuran as a solvent to afford montelukast sodium.

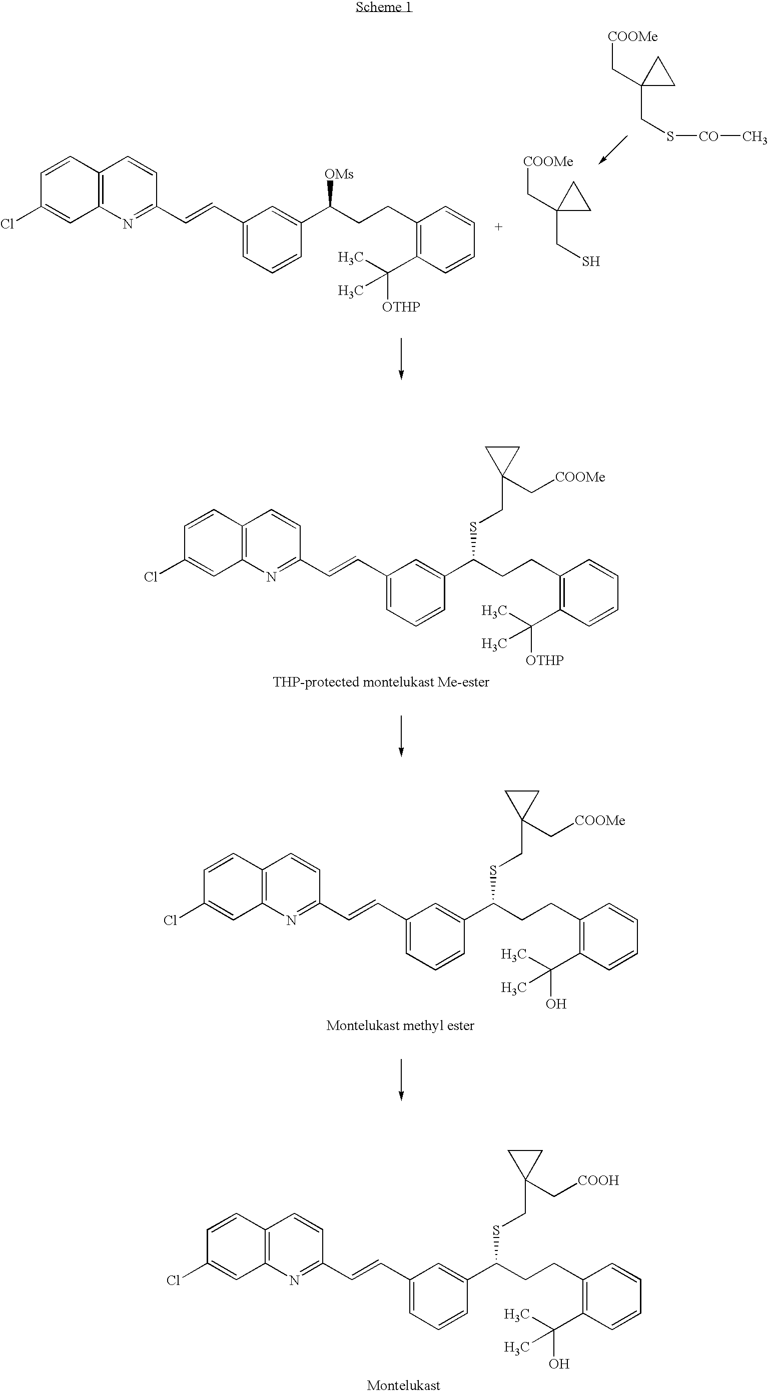

Many other synthetic schemes are proposed in U.S. Pat. No. 5,565,473 for making montelukast and/or other compounds.

In WO 95/18107 another approach has been applied, here a crystalline alkyl- or aryl-sulfonate intermediate compound, preferably a methane sulfonate compound, is reacted with a dilithium anion of 1-(mercaptomethyl)cyclopropane-1-acetic acid as represented in Scheme 2

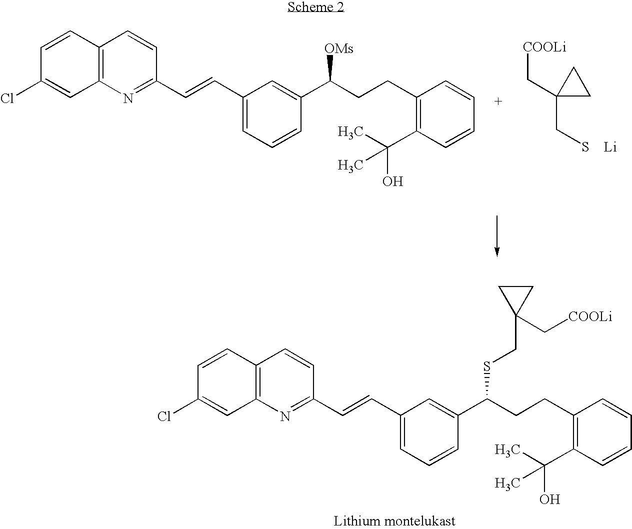

There are several other processes reported in the prior art for the preparation of montelukast and its salts. U.S. Pat. No. 5,614,632 discloses a process for the preparation of the sodium salt of montelukast and certain process intermediates. The process involves generation of dilithium dianion of 1-(mercaptomethyl)cyclopropaneacetic acid followed by condensation with 2-(2-(3(S)-(3-(2-(7-chloro-2-quinolinyl)ethenyl)phenyl)-3-methanesulfonyloxypropyl)phenyl)-2-propanol (referred as mesylated alcohol) to afford montelukast, which is further converted to the corresponding sodium salt via dicyclohexyl amine salt. The '362 patent also discloses a process for the preparation of crystalline montelukast sodium salt and mesylated alcohol. The process involves reacting methyl 2(3(S)-(3-(2-(7-chloro-2-quinolinyl)ethenyl)phenyl)-3-hydroxy propyl)benzoate with methyl magnesium chloride to give a diol, which is further converted to mesylated alcohol on reaction with methane sulfonyl chloride as shown in Scheme 3.

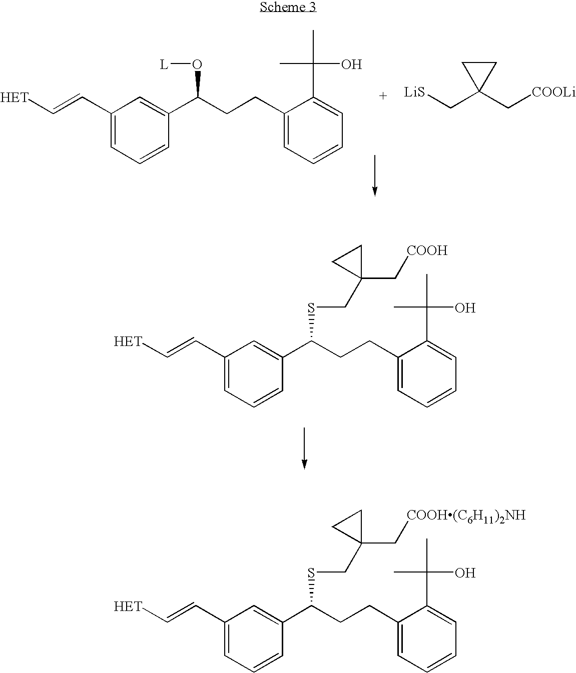

US20050107612 discloses a process for preparation of montelukast or a salt wherein methyl-2-(3-(3-(2-(7-chloro-2-quinolinyl)ethenyl)phenyl)-3-oxopropyl benzoate of Formula (II) is reduced with (+) B-chloro diisopinocampheylborane as a chiral reducing reagent in polar organic solvents to afford methyl-2-(3-(3-(2-(7-chloro-2-quinoliny)pethenyl)phenyl)-3-hydroxy propyl benzoate of Formula (III). The compound of formula (III) is mesylated with methane sulfonyl chloride or tosylated with toluene sulfonyl chloride to form methyl-2-(3-(3-(2-(7-chloro-2-quinoliny)pethenyl)phenyl)-3-methane sulfonyloxy propyl benzoate of formula (IV) or a corresponding tosylate. (IV)) is then condensed with 1-mercapto methyl cyclopropane acetic acid of formula (V) in the presence of a base. The product of this reaction is preferably isolated in the form of an organic amine salt of formula (VI), preferably, dicyclohexyl amine salt. The resultant amine salt is reacted with methyl magnesium chloride or methyl magnesium bromide in an organic solvent to get montelukast free acid and is again converted to its organic amine salt of formula (VII) to get more pure compound. The amine salt of montelukast of formula (VII) is conveniently converted into pharmaceutically acceptable salts, preferably sodium salt using sodium methoxide or sodium hydroxide (Scheme 4).

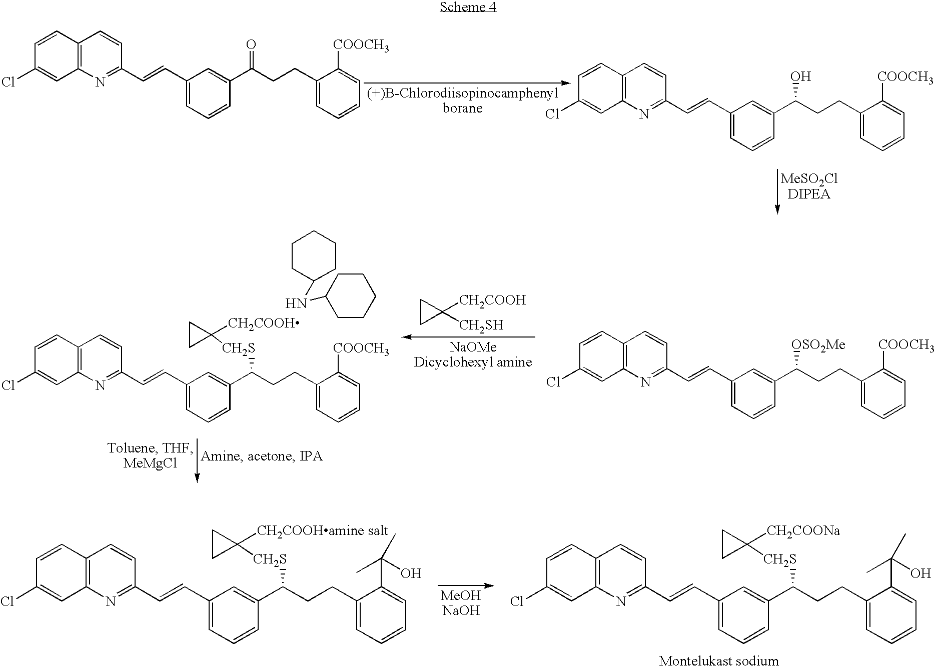

The above-processes, however, have various drawbacks and it would be desirable to provide a different, useful process for making montelukast and its salts, especially a process that could be suitable for use on an industrial scale.

### SUMMARY OF THE INVENTION

According to the present invention, there is provided an improved process for preparation of montelukast and its salts via novel intermediates.

According to one aspect of the invention, there is provided a process for preparing a compound of formula 2:

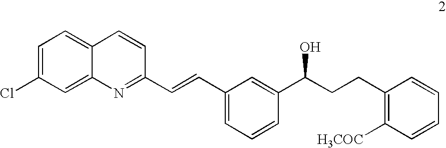

comprising reacting a compound of formula 1:

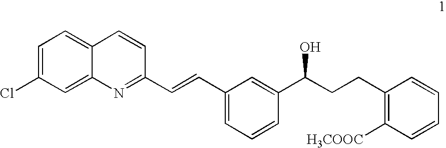

with lithium hexamethyldisilazide (LiHMDS) or N,O-dimethylhydroxylamine hydrochloride followed by reaction with methyl magnesium halide (MeMgX).

According to another aspect of the invention there is provided a process for preparing a compound of formula 3:

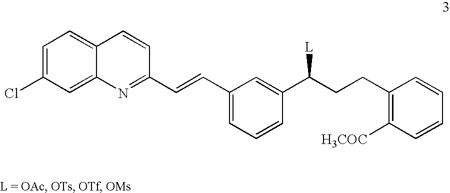

comprising reacting a compound of formula 2:

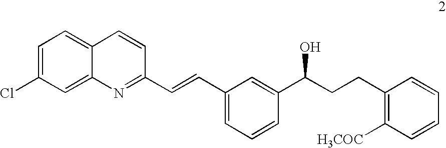

with an appropriate triflate, tosylate, acetate or mesylate compound.

According to another aspect of the invention there is provided a process for preparing a compound of formula 4, or a salt thereof:

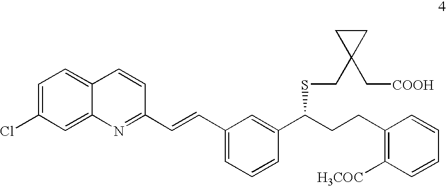

comprising reacting a compound of formula 3:

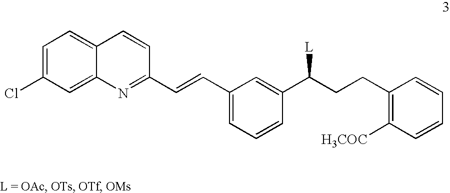

with 1-(mercaptomethyl)cyclopropane acetic acid in the presence of a base and an inert solvent. Optionally, compound 4 can be converted to an appropriate salt, such as an organic amine salt.

According to another aspect of the invention there is provided a process for preparing a compound of formula 6:

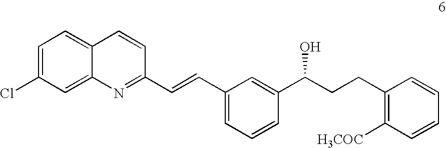

comprising reacting a compound of formula 5:

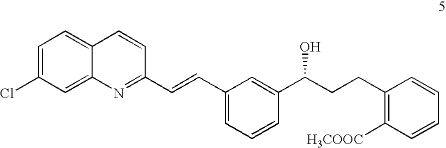

with lithium hexamethyldisilazide (LiHMDS) or N,O-dimethylhydroxylamine hydrochloride followed by reaction with methyl magnesium halide (MeMgX).

According to another aspect of the invention there is provided a process for preparing a compound of formula 7:

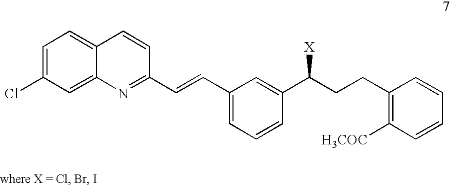

comprising reacting a compound of formula 2:

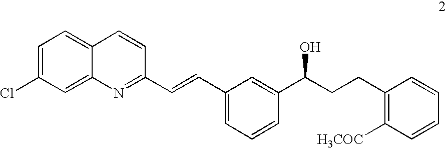

with a halogen donating compound.

According to another aspect of the invention there is provided a process for preparing a compound of formula 7:

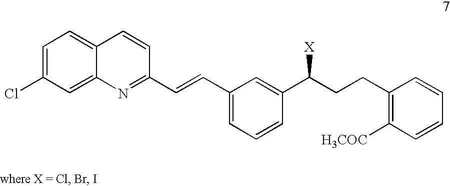

comprising reacting a compound of formula 6:

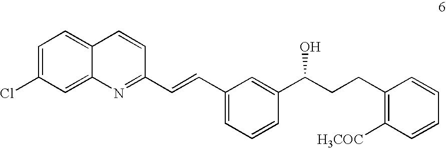

with a halogen donating compound.

According to another aspect of the invention there is provided a process for preparing a compound of formula 4, or a salt thereof:

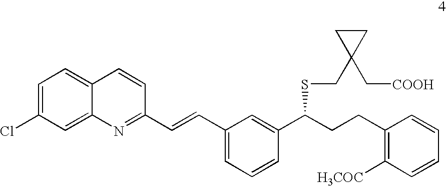

comprising reacting a compound of formula 7:

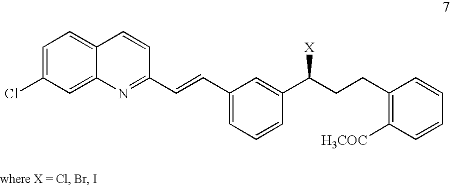

with 1-(mercaptomethyl)cyclopropane acetic acid in the presence of a base and an inert solvent. Optionally, compound 4 can be converted to an appropriate salt, such as an organic amine salt.

According to another aspect of the invention there is provided a process for preparing a compound of formula I (montelukast), or a salt thereof:

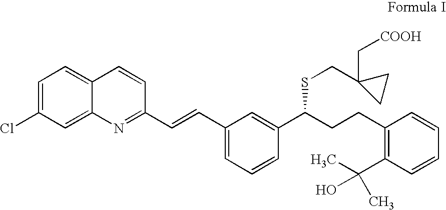

comprising reacting a compound of formula 4, or a salt thereof:

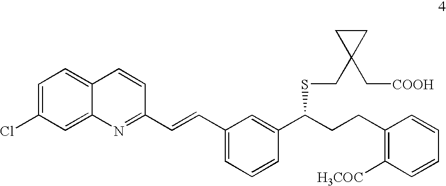

with methyl magnesium halide in the presence of an inert solvent.

In accordance with the invention there is also provided compounds, 2, 3, 4, 6 and 7:

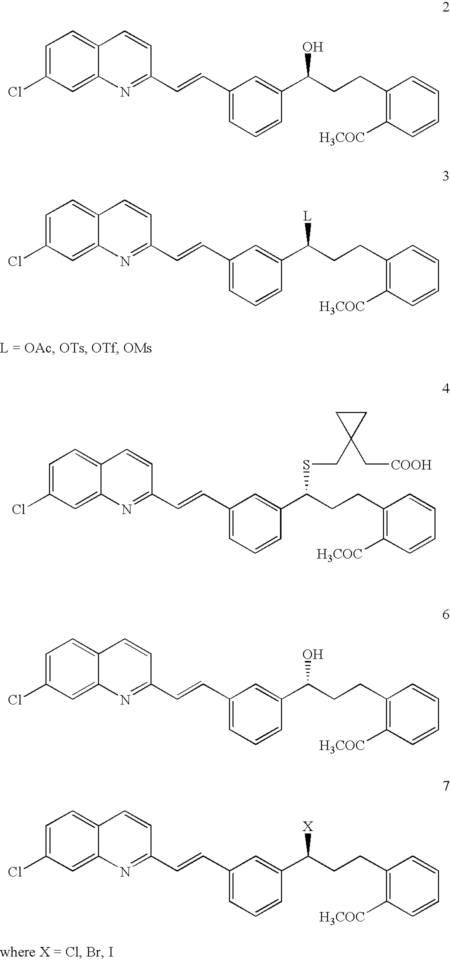

Compounds 4 and montelukast may be provided in salt form. Salts of particular interest include an organic amine salt of compound 4, the organic amine salt of montelukast and the sodium salt of montelukast. In one preferred embodiment, the compound (4) is isolated as the organic amine salt, then converted to the organic amine salt of montelukast, then converted to the sodium salt of montelukast. One preferred organic amine salt is the dicyclohexylamine salt.

### DETAILED DESCRIPTION OF THE INVENTION

The present invention provides a process for the synthesis of montelukast and its salts involving novel compounds of formula (3) with a group L where (L=OAc OTs and OTf) as shown in scheme 5.

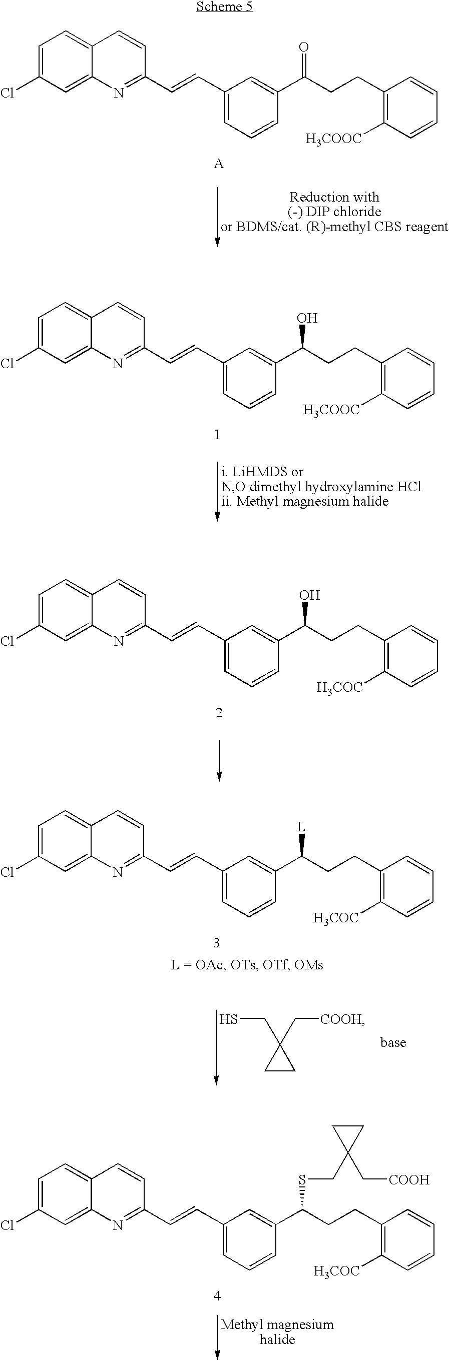

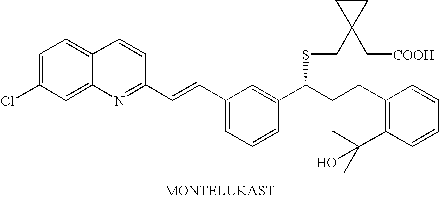

The process of the present invention comprises reacting compound A with a chiral reducing agent like (−)DIP chloride (diisopinocampheylchloroborane) or with (borane dimethyl sulfide) BDMS and cat. (R)-CBS reagent to obtain the (S)-alcohol (1). Compound (1) is then reacted with lithium hexamethyldisilazide (LiHMDS) or N,O-dimethylhydroxylamine hydrochloride followed by reaction with methyl magnesium halide (MeMgX where X=Cl, Br) to give compound (2). Compound (2) is converted to compound (3) having group L (where L=OTf, OTs, OAc, OMs) by reaction with trifluoromethane sulfonic anhydride (triflic anhydride) p-toluene sulfonyl chloride (tosyl chloride), acetic anhydride, methane sulphonyl chloride to obtain the triflate (i.e., trifluoromethanesulfonate, ‘OTf’), tosylate (OTs) and acetate (OAc) and mesylate (OMs), to obtain the compounds (3) respectively.

In the process of the present invention the trifluoromesylation, tosylation and acetylation can be performed on compound (2) with the above mentioned reagents at temperatures ranging from −10 to 50° C., more preferably −5 to 10° C. for addition and warming to room temperature in a water-free inert solvent. The solvents may be chosen from THF, diethyl ether, 1,4 dioxane, toluene, benzene, Methylene dichloride, chloroform and the like. A tertiary base like trimethyl amine, triethyl amine, pyridine, N,N, diisopropyl ethyl amine (DIPEA) and the like can be added to assist the reaction. A catalytic amount of 4, 4 dimethyl amino pyridine (DMAP) can also be added to enhance the reaction. The reaction is completed within 1 to 24 hrs after the addition, preferably after overnight stirring.

In another aspect the compound (3) is further converted into compound (4) by reaction with 1-(mercaptomethyl)cyclopropane acetic acid in presence of a base like sodium hydride, sodamide, cesium carbonate, sodium methoxide, potassium tert-butoxide and the like in an inert solvent like THF, DMF, dioxane, N-methylpyrrolidone (NMP) and the like at temperatures ranging from −10 to 50° C.

In yet another aspect compound (4) on treatment with MeMgX in presence of an inert solvent like toluene, THF, dioxane, dichloromethane, chloroform etc. gives the title compound montelukast (I). The reaction can be performed at temperatures ranging from −10 to 50° C. more preferably 0-10° C. The reaction time may vary from 1 to 24 hrs, preferably in 5-10 hrs. After completion of the reaction, montelukast (I) is isolated from the reaction mass in a conventional manner.

Another aspect of the present invention involves preparation of novel compounds of formula (7) where X=(Cl, Br, I) by reacting the compound (A) with a halogen donating substituent to obtain compound (2) and its isomer (6) as shown in scheme 6. Compound (2) can be made to undergo with retention of configuration to obtain compound (7)—this can be achieved by carrying out the reaction in the presence of a solvent. Similarly, compound (6) undergoes inversion of configuration to produce compound (7)—this can be achieved by carrying out the reaction in the absence of a solvent.

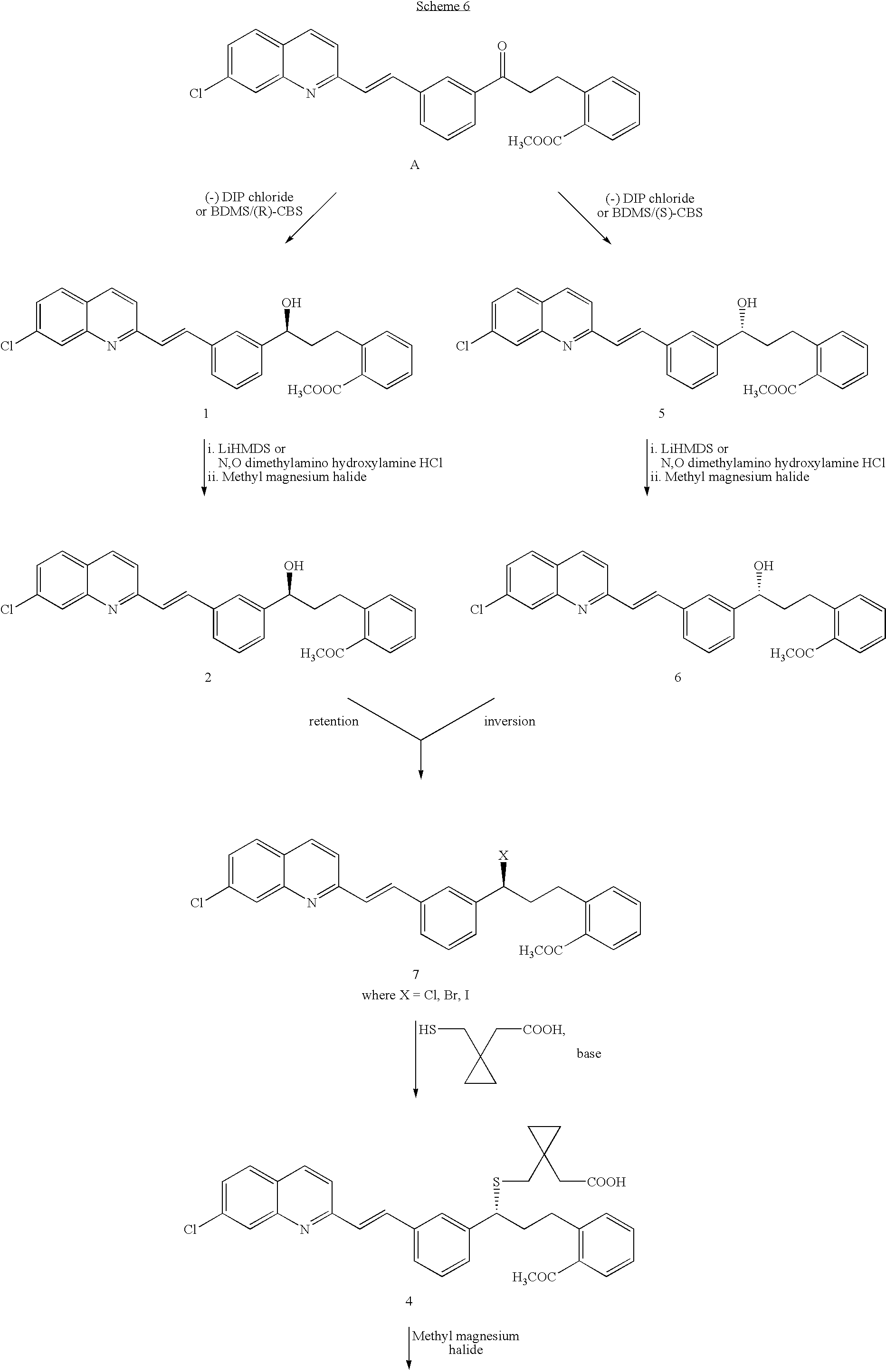

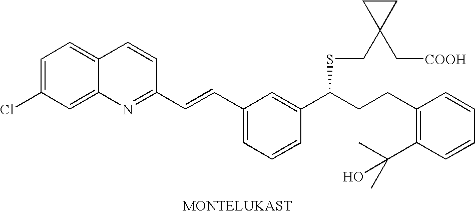

The alternative synthesis for the preparation of montelukast as described in scheme 6, wherein compound A is chirally reduced to obtain the (S)—alcohol (1) and (R)-alcohol (5) by using (−) and (+) diisopinocamphenyl borane chloride (DIP chloride) respectively or borane dimethyl sulfide (BDMS) with catalytic (R)-methyl CBS reagent and (S)-methyl CBS reagent respectively.

The (S) alcohol (1) and (R) alcohol (5) are then treated with lithium hexamethyldisilazide (LiHMDS) or N,O-dimethylhydroxylamine hydrochloride followed by reaction with methyl magnesium halide to obtain the ketones (2) and (6) respectively.

Ketones (2) and (6) are treated with halogen donating compound to obtain the desired (S)-halo compound (7).

Chlorination can be performed on compound (2) with retention of configuration with thionyl chloride, phosphorus pentachloride, phosphorus oxychloride, most preferably thionyl chloride in presence of an inert solvent at 0-50° C. This reaction can also be carried out optionally in the presence of an organic base like pyridine, trimethyl amine, triethyl amine.

Bromination can be performed on compound (2) with any of the following: N-bromo succinimide, bromine, phosphorus oxybromide, phosphorus tribromide and the like, most preferably N-bromo succinimide can be used in an inert solvent or without solvent at 0-50° C. The reaction requires 1 to 24 hrs for completion or more preferably 5 to 10 hrs. The addition of the reagent can be done at 0 to 10° C. and warmed to the requisite temperature for completion.

Iodination can be done with conversion of the hydroxyl group on compound (2) to trialkylsilyloxy group most conveniently done with trimethylsilyl halide and reacting the same with an alkali metal iodide like potassium iodide, sodium iodide or cesium iodide or can be reacted with tetrabutyl ammonium iodide and the like. The silylation of the hydroxyl group can be done at −10° C. to 25° C. in presence of a tertiary base like, triethyl amine, pyridine, DIPEA and the like. After completion of silylation, the compound is reacted with the alkali metal iodide most preferably sodium or potassium iodide at temperatures ranging from 0 to 100° C. Polar aprotic solvents like THF, DMF. DMA, DMSO and the like can be used for performing the reaction. Chlorinated and aromatic solvents can however be also used for the reaction.

Compound (6) is treated with a halide donating compound so as to obtain compound (7) thus leading to inversion of configuration as shown in scheme 6. Thus chlorination with inversion of configuration can be done for e.g. with thionyl chloride in absence of any solvent, the reaction is carried out neat without using any solvent. The reaction can be carried out optionally in the presence of a organic base like pyridine, trimethyl amine, triethyl amine and the like to obtain the (R)-alcohol (6). The time and temperature for the reaction are similar to as mentioned above. Bromination can be done with thionyl bromide in presence of an organic tertiary base or with bromine and a tertiary base. Iodination can be performed on compound (6) under Mitsunobu conditions i.e. with triphenylphosphine and diethyl azadicarboxylate (DEAD) and reacting with alkali metal iodide to obtain compound 7

Once compound (7) is prepared, it can be made to react similarly as compound (3) in the earlier aspect of the present invention to obtain compound (4) which is then converted to the title compound montelukast (I).

The invention will now be further described with reference to the following examples.

### Example 1

### Preparation of Compound (1)

Methylene chloride (200 ml) and Compound (A) (40 gm) was charged, cooled to −5° C., Diisopropylethylamine (28 ml) was added followed by (−) DIP chloride (120 ml) at −5 to 0° C. over a period of 45 minutes. The temperature of the reaction mass was maintained at −5 to 0° C. for 3-4 hours, after completion of the reaction, triethanolamine (14.4 gm) was added at less than 20° C., stirred at 25-30° C. for 2 hours. Separated methylene chloride layer, extracted the aqueous layer with methylene chloride (100 ml), combined methylene chloride layers and washed with 10% of sodium chloride solution (100 ml). Distilled off methylene chloride completely under vacuum at less than 35° C. to residue. The residue was dissolved in methanol (480 ml), stirred for 1 hour, clarified, added water (200 ml) slowly over a period of 1 hour, stirred at room temperature for 2 hours, filtered, washed with methanol-water mixture (20 ml each). The material so obtained was stirred with heptane (160 ml) for 45 minutes, filtered, washed with heptane (40 ml) and dried the material under vacuum at 45-50° C. to give Compound (1) (39 gm, 97% yield, 98% HPLC purity).

### Example 2

### Preparation of Compound (2)

Lithium hexamethyldisilazide (314 ml-28% solution in hexane) was charged under nitrogen atmosphere, chilled to −5 to 0° C., 3 M solution of methyl magnesium chloride (87.3 ml) was added slowly over a period of 30 minutes under nitrogen atmosphere at −5 to 0° C. The contents were stirred for 15 minutes at −5 to 0° C. under nitrogen atmosphere and a solution of Compound (1) in THF (20 gm in 200 ml) was slowly added over a period of 30 minutes, stirred at −5 to 0° C. for 5 hours, adjusted the pH of the reaction mass to neutral using 10% acetic acid below 10° C., extracted using ethyl acetate (200 ml×2), washed ethyl acetate layer with water (100 ml), dried over sodium sulphate (5 gm) and distilled out ethyl acetate under vacuum at 40-45° C. to give Compound (2) as residue (15.4 gm, 80% yield, 80% HPLC purity).

### Example 3

### Preparation of Acetyl Derivative of Compound (2)

Acetic anhydride (22.3 gm) and glacial acetic acid (34.3 gm) was added followed by Compound (2) (25 gm) at room temperature. To this perchloric acid (2 ml) was added at 25-30° C., the contents were stirred at 30-35° C. for 2 hours, quenched the reaction mass with ice-water mixture (250 ml). Extracted the aqueous layer with ethyl acetate (250 ml), washed with 5% of sodium bicarbonate solution (75 ml) followed by water (75 ml), dried over sodium sulphate (2.5 gm) and distilled off ethyl acetate completely under vacuum at less than 40° C. to give acetyl derivative of compound (2) (24 gm, 87.9% yield, 83% HPLC purity).

### Example 4

### Preparation of Tosyl Derivative of Compound (2)

Compound (2) (25 gm) and toluene (250 ml) was charged under nitrogen atmosphere, pyridine (25 ml) was added, cooled to 5-10° C., a solution of para toluene sulphonyl chloride in toluene (13 gm in 125 ml) was added dropwise at 5-10° C., maintained for 30 minutes then heated to 55-60° C., maintained for 3 hours and distilled out toluene completely under vacuum at 45-50° C. To this ice-water mixture (250 ml) was added, pH of the reaction mass was adjusted to neutral using 1% acetic acid, extracted with ethyl acetate (250 ml), ethyl acetate layer was washed with water (75 ml), dried over sodium sulphate (2.5 gm) and distilled out ethyl acetate completely under vacuum to give Tosyl derivative of compound (2) (26 gm, 77% yield, 78% HPLC purity).

### Example 5

### Preparation of Triflic Derivative of Compound (2)

Compound (2) (25 gm) and methylene chloride (250 ml) was charged under nitrogen atmosphere, pyridine (5 ml) was added, triflic anhydride (32 gm) was added at 0-5° C. and the contents were stirred at 25-30° C. for 2 hours. Water (50 ml) was added, stirred for 30 minutes, separated the layers, the aqueous layer was extracted with methylene chloride (25 ml), the combined methylene chloride layer was washed with water (25 ml), dried over sodium sulphate (2.5 gm) and distilled off methylene chloride completely under vacuum at less than 35° C. to give Triflic derivative of compound (2) (30 gm, 93% yield, 87% HPLC purity).

### Example 6

### Preparation of Keto-Montelukast-Dicyclohexylamine Salt [Compound (4)-DCHA Salt] from Acetyl Derivative of Compound (2)

Dimethyl formamide (100 ml) and Cesium carbonate (70.83 gm) was charged at room temperature under nitrogen atmosphere, heated to 60-62° C. and maintained for 15 minutes. The contents were chilled to 15-20° C., a solution of 1-(mercaptomethyl)cyclopropane acetic acid in DMF (9.04 gm in 50 ml) was added slowly to the reaction mass at 15-20° C. over a period of 30 minutes and maintained for 1 hour. The temperature of the reaction mass was raised to room temperature and a solution of acetyl derivative of Compound (2) in DMF (25 gm in 100 ml) was added slowly over a period of 15-30 minutes, the reaction mass was heated to 35-40° C. and maintained for 2 hours. The reaction mass was filtered, washed with methanol (12.5 ml), water (300 ml) was charged to the clear filtrate and washed with toluene (125 ml×2). The pH of the aqueous layer was adjusted to 5.0-6.0 using 10% acetic acid solution and extracted with ethyl acetate (125 ml×2). The ethyl acetate layer was washed with water (125 ml) followed by 10% of sodium chloride solution (125 ml), dried over sodium sulphate (5 gm) and distilled out ethyl acetate completely under vacuum at 40-45° C. to residue. Ethyl acetate (100 ml) and Dicyclohexylamine (10.5 gm) was charged to residue at room temperature, stirred for 2 hours, chilled the contents to 0-5° C., filtered, washed with chilled ethyl acetate (25 ml) and dried under vacuum at 40-45° C. to give Keto-montelukast-Dicyclohexylamine salt (12.0 gm, 32% yield, 97% HPLC purity).

### Example 7

### Preparation of Keto-Montelukast-Dicyclohexylamine Salt [Compound (4)-DCHA Salt] from Tosyl Derivative of Compound (2)

Dimethyl formamide (200 ml) and Cesium carbonate (141.66 gm) was charged at room temperature under nitrogen atmosphere, heated to 60-62° C. and maintained for 15 minutes. The contents were chilled to 15-20° C., a solution of 1-(mercaptomethyl)cyclopropane acetic acid in DMF (17.4 gm in 100 ml) was added slowly to the reaction mass at 15-20° C. over a period of 30 minutes and maintained for 1 hour. The temperature of the reaction mass was raised to room temperature and a solution of tosyl derivative of Compound (2) in DMF (50 gm in 200 ml) was added slowly over a period of 15-30 minutes, the reaction mass was heated to 35-40° C. and maintained for 2 hours. The reaction mass was filtered, washed with methanol (25 ml), water (600 ml) was charged to the clear filtrate and washed with toluene (250 ml×2). The pH of the aqueous layer was adjusted to 5.0-6.0 using 10% acetic acid solution and extracted with ethyl acetate (250 ml×2). The ethyl acetate layer was washed with water (250 ml) followed by 10% of sodium chloride solution (250 ml), dried over sodium sulphate (10 gm) and distilled out ethyl acetate completely under vacuum at 40-45° C. to residue. Ethyl acetate (200 ml) and Dicyclohexylamine (18.4 gm) was charged to residue at room temperature, stirred for 2 hours, chilled the contents to 0-5° C., filtered, washed with chilled ethyl acetate (50 ml) and dried under vacuum at 40-45° C. to give Keto-montelukast-Dicyclohexylamine salt (17.4 gm, 30% yield, 95% HPLC purity).

### Example 8

### Preparation of Keto-Montelukast-Dicyclohexylamine Salt [Compound (4)-DCHA Salt] from Triflic Derivative of Compound (2)

Dimethyl formamide (40 ml) and Cesium carbonate (28.4 gm) was charged at room temperature under nitrogen atmosphere, heated to 60-62° C. and maintained for 15 minutes. The contents were chilled to 15-20° C., a solution of 1-(mercaptomethyl)cyclopropane acetic acid in DMF (3.04 gm in 20 ml) was added slowly to the reaction mass at 15-20° C. over a period of 30 minutes and maintained for 1 hour. The temperature of the reaction mass was raised to room temperature and a solution of triflic derivative of Compound (2) in DMF (10 gm in 40 ml) was added slowly over a period of 15-30 minutes, the reaction mass was heated to 35-40° C. and maintained for 2 hours. The reaction mass was filtered, washed with methanol (5 ml), water (120 ml) was charged to the clear filtrate and washed with toluene (50 ml×2). The pH of the aqueous layer was adjusted to 5.0-6.0 using 10% acetic acid solution and extracted with ethyl acetate (50 ml×2). The ethyl acetate layer was washed with water (50 ml) followed by 10% of sodium chloride solution (50 ml), dried over sodium sulphate (2.5 gm) and distilled out ethyl acetate completely under vacuum at 40-45° C. to residue. Ethyl acetate (40 ml) and Dicyclohexylamine (3.5 gm) was charged to residue at room temperature, stirred for 2 hours, chilled the contents to 0-5° C., filtered, washed with chilled ethyl acetate (10 ml) and dried under vacuum at 40-45° C. to give Keto-montelukast-Dicyclohexylamine salt (4.5 gm, 35% yield, 96.5% HPLC purity).

### Example 9

### Preparation of Compound (7) [Chloro Compound]

Methylene chloride (200 ml) and Compound (2) (20 gm) was charged at room temperature under nitrogen atmosphere, chilled to 0-5° C. and thionyl chloride (16.2 gm) was added slowly to the reaction mass at 0-5° C. under nitrogen atmosphere over a period of 1 hour. The contents were stirred at 0-5° C. for 3-4 hours and distilled out methylene chloride completely under vacuum at less than 35° C. to residue. The residue was dissolved in methylene chloride (200 ml) was quenched into ice-water mixture (300 ml) below 15° C. over a period of 30 minutes. The pH of the reaction mass was adjusted to neutral using 10% sodium bicarbonate solution, separated methylene chloride layer, the aqueous layer was extracted with methylene chloride (100 ml), washed the combined methylene chloride layer with water (100 ml), dried over sodium sulphate (5 gm) and distilled out methylene chloride completely under vacuum at 35-40° C. to give Compound (7) (20 gm, 95.7% yield, 85% HPLC purity).

### Example 10

### Preparation of Compound (5)

Methylene chloride (100 ml) and Compound (A) (20 gm) was charged, cooled to −5° C., Diisopropylethylamine (14 ml) was added followed by (+) DIP chloride (60 ml) at −5 to 0° C. over a period of 45 minutes. The temperature of the reaction mass was maintained at −5 to 0° C. for 3-4 hours, after completion of the reaction, triethanolamine (7.2 gm) was added at less than 20° C., stirred at 25-30° C. for 2 hours. Separated methylene chloride layer, extracted the aqueous layer with methylene chloride (50 ml), combined methylene chloride layers and washed with 10% of sodium chloride solution (50 ml). Distilled off methylene chloride completely under vacuum at less than 35° C. to residue. The residue was dissolved in methanol (240 ml), stirred for 1 hour, clarified, added water (100 ml) slowly over a period of 1 hour, stirred at room temperature for 2 hours, filtered, washed with methanol-water mixture (10 ml each). The material so obtained was stirred with heptane (80 ml) for 45 minutes, filtered, washed with heptane (20 ml) and dried the material under vacuum at 45-50° C. to give Compound (5) (18.5 gm, 92% yield, 97.5% HPLC purity).

### Example 11

### Preparation of Compound (6)

Lithium hexamethyldisilazide (157 ml-28% solution in hexane) was charged under nitrogen atmosphere, chilled to −5 to 0° C., 3 M solution of methyl magnesium chloride (44 ml) was added slowly over a period of 30 minutes under nitrogen atmosphere at −5 to 0° C. The contents were stirred for 15 minutes at −5 to 0° C. under nitrogen atmosphere and a solution of Compound (5) in THF (10 gm in 100 ml) was slowly added over a period of 30 minutes, stirred at −5 to 0° C. for 5 hours, adjusted the pH of the reaction mass to neutral using 10% acetic acid below 10° C., extracted using ethyl acetate (100 ml×2), washed ethyl acetate layer with water (50 ml), dried over sodium sulphate (2.5 gm) and distilled out ethyl acetate under vacuum at 40-45° C. to give Compound (6) (7.5 gm, 78% yield, 79.9% HPLC purity).

### Example 12

### Preparation of Compound (7) [Chloro Compound] from Compound (6)

To 20 gms of Compound (6) phosphorus oxychloride (50 ml) was added slowly under nitrogen atmosphere and stirred at room temperature for 2 hours. After completion of the reaction, methylene chloride (100 ml) was added and stirred to get clear solution. The organic layer was washed with 5% of sodium bicarbonate (20 ml), dried over sodium sulphate and distilled off methylene chloride to give Compound (7) [Chloro compound] (18.7 gm, 90% yield, 78.5% HPLC purity).

### Example 13

### Preparation of Keto-Montelukast Salts. Compound (4)

### a) Preparation of Keto-Montelukast-Dicyclohexylamine Salt [Compound (4)-DCHA Salt]

Dimethyl formamide (100 ml) and Cesium carbonate (70.83 gm) was charged at room temperature under nitrogen atmosphere, heated to 60-62° C. and maintained for 15 minutes. The contents were chilled to 15-20° C., a solution of 1-(mercaptomethyl)cyclopropane acetic acid in DMF (9.52 gm in 50 ml) was added slowly to the reaction mass at 15-20° C. over a period of 30 minutes and maintained for 1 hour. The temperature of the reaction mass was raised to room temperature and a solution of Compound (7) in DMF (25 gm in 100 ml) was added slowly over a period of 15-30 minutes, the reaction mass was heated to 35-40° C. and maintained for 2 hours. The reaction mass was filtered, washed with methanol (12.5 ml), water (300 ml) was charged to the clear filtrate and washed with toluene (125 ml×2). The pH of the aqueous layer was adjusted to 5.0-6.0 using 10% acetic acid solution and extracted with ethyl acetate (125 ml×2). The ethyl acetate layer was washed with water (125 ml) followed by 10% of sodium chloride solution (125 ml), dried over sodium sulphate (5 gm) and distilled out ethyl acetate completely under vacuum at 40-45° C. to residue. Ethyl acetate (100 ml) and Dicyclohexylamine (10.8 gm) was charged to residue at room temperature, stirred for 2 hours, chilled the contents to 0-5° C., filtered, washed with chilled ethyl acetate (25 ml) and dried under vacuum at 40-45° C. to give Keto-montelukast-Dicyclohexylamine salt (12.5 gm, 34.4% yield, 98% HPLC purity).

### b) Preparation of Keto-Montelukast-Isopropylamine Salt [Compound (4) —IPA Salt]

Dimethyl formamide (200 ml) and Cesium carbonate (141.66 gm) was charged at room temperature under nitrogen atmosphere, heated to 60-62° C. and maintained for 15 minutes. The contents were chilled to 15-20° C., a solution of 1-(mercaptomethyl)cyclopropane acetic acid in DMF (19.04 gm in 100 ml) was added slowly to the reaction mass at 15-20° C. over a period of 30 minutes and maintained for 1 hour. The temperature of the reaction mass was raised to room temperature and a solution of Compound (7) in DMF (50 gm in 200 ml) was added slowly over a period of 15-30 minutes, the reaction mass was heated to 35-40° C. and maintained for 2 hours. The reaction mass was filtered, washed with methanol (25 ml), water (600 ml) was charged to the clear filtrate and washed with toluene (250 ml×2). The pH of the aqueous layer was adjusted to 5.0-6.0 using 10% acetic acid solution and extracted with ethyl acetate (250 ml×2). The ethyl acetate layer was washed with water (250 ml) followed by 10% of sodium chloride solution (250 ml), dried over sodium sulphate (10 gm) and distilled out ethyl acetate completely under vacuum at 40-45° C. to residue. Ethyl acetate (200 ml) and Isopropylamine (7.04 gm) was charged to residue at room temperature, stirred for 2 hours, chilled the contents to 0-5° C., filtered, washed with chilled ethyl acetate (50 ml) and dried under vacuum at 40-45° C. to give Keto-montelukast-isopropylamine salt (22.0 gm, 31.4% yield, 96.9% HPLC purity).

### c) Preparation of Keto-Montelukast-Cyclohexylamine Salt [Compound (4) —CHA Salt]

Dimethyl formamide (100 ml) and Cesium carbonate (70.83 gm) was charged at room temperature under nitrogen atmosphere, heated to 60-62° C. and maintained for 15 minutes. The contents were chilled to 15-20° C., a solution of 1-(mercaptomethyl)cyclopropane acetic acid in DMF (9.52 gm in 50 ml) was added slowly to the reaction mass at 15-20° C. over a period of 30 minutes and maintained for 1 hour. The temperature of the reaction mass was raised to room temperature and a solution of Compound (7) in DMF (25 gm in 100 ml) was added slowly over a period of 15-30 minutes, the reaction mass was heated to 35-40° C. and maintained for 2 hours. The reaction mass was filtered, washed with methanol (12.5 ml), water (300 ml) was charged to the clear filtrate and washed with toluene (125 ml×2). The pH of the aqueous layer was adjusted to 5.0-6.0 using 10% acetic acid solution and extracted with ethyl acetate (125 ml×2). The ethyl acetate layer was washed with water (125 ml) followed by 10% of sodium chloride solution (125 ml), dried over sodium sulphate (5 gm) and distilled out ethyl acetate completely under vacuum at 40-45° C. to residue. Ethyl acetate (100 ml) and Cyclohexylamine (5.9 gm) was charged to residue at room temperature, stirred for 2 hours, chilled the contents to 0-5° C., filtered, washed with chilled ethyl acetate (25 ml) and dried under vacuum at 40-45° C. to give Keto-montelukast-cyclohexylamine salt (12 gm, 33.1% yield, 97.4% HPLC purity).

### Example 14

### Preparation of Montelukast Salts

### a) Preparation of Montelukast-Isopropylamine Salt

Part 1: Keto-montelukast-Dicyclohexylamine salt (10 gm), water (100 ml) and Methylene chloride (100 ml) was charged under nitrogen atmosphere, adjusted the pH of the reaction mass to 5.0-6.0 using 10% acetic acid solution, stirred for 10 minutes and separated the methylene chloride layer. Aqueous layer was extracted with methylene chloride (50 ml), combined methylene chloride layer was dried over sodium sulphate (2.5 gm), distilled off methylene chloride completely under vacuum below 40° C. and stripped off methylene chloride with toluene (50 ml×2) to residue. The residue keto-montelukast was dissolved in toluene (20 ml) and used in part 2.

Part 2: THF (100 ml) and Cerium chloride (3.6 gm) was charged under nitrogen atmosphere, heated the contents to reflux and maintained for 3 hours. It was then, chilled to −5° C. and 3M methyl magnesium chloride (36 ml) was added dropwise over a period of 45 minutes and maintained at 0-5° C. for 2 hours. To this part 1 solution was added at −5 to 0° C. over a period of 1 hour and maintained for 1 hours. After completion of reaction, the mass was quenched into ice-water mixture (100 ml), pH of the reaction mass was adjusted to 6.0-6.5 using 10% acetic acid solution and extracted with ethyl acetate (50 ml×2). The ethyl acetate layer was dried over sodium sulphate (5 gm) and distilled off ethyl acetate completely under vacuum at less than 45° C. to residue. The residue was dissolved in ethyl acetate (40 ml), Isopropylamine (0.86 gm) was added, stirred for 1 hour, cooled to 0-5° C. and maintained for 1 hour. The material was filtered, washed with chilled ethyl acetate (5 ml) and dried under vacuum at 40-45° C. to give Montelukast-Isopropylamine salt (6.0 gm, 70% yield, 99% HPLC purity).

### b) Preparation of Montelukast-Morpholine Salt

Part 1: Keto-montelukast-Dicyclohexylamine salt (5 gm), water (50 ml) and Methylene chloride (50 ml) was charged under nitrogen atmosphere, adjusted the pH of the reaction mass to 5.0-6.0 using 10% acetic acid solution, stirred for 10 minutes and separated the methylene chloride layer. Aqueous layer was extracted with methylene chloride (25 ml), combined methylene chloride layer was dried over sodium sulphate (1.25 gm), distilled off methylene chloride completely under vacuum below 40° C. and stripped off methylene chloride with toluene (25 ml×2) to residue. The residue keto-montelukast was dissolved in toluene (10 ml) and used in part 2.

Part 2: THF (50 ml) and Cerium chloride (1.8 gm) was charged under nitrogen atmosphere, heated the contents to reflux and maintained for 3 hours. It was then, chilled to −5° C. and 3M methyl magnesium chloride (18 ml) was added dropwise over a period of 45 minutes and maintained at 0-5° C. for 2 hours. To this part 1 solution was added at −5 to 0° C. over a period of 1 hour and maintained for 1 hours. After completion of reaction, the mass was quenched into ice-water mixture (50 ml), pH of the reaction mass was adjusted to 6.0-6.5 using 10% acetic acid solution and extracted with ethyl acetate (25 ml×2). The ethyl acetate layer was dried over sodium sulphate (2.5 gm) and distilled off ethyl acetate completely under vacuum at less than 45° C. to residue. The residue was dissolved in ethyl acetate (20 ml), Morpholine (0.63 gm) was added, stirred for 1 hour, cooled to 0-5° C. and maintained for 1 hour. The material was filtered, washed with chilled ethyl acetate (2.5 ml) and dried under vacuum at 40-45° C. to give Montelukast-morpholine salt (3.0 gm, 67.4% yield, 98.6% HPLC purity).

### c) Preparation of Montelukast-Cyclohexylamine Salt

Part 1: Keto-montelukast-Dicyclohexylamine salt (20 gm), water (200 ml) and Methylene chloride (200 ml) was charged under nitrogen atmosphere, adjusted the pH of the reaction mass to 5.0-6.0 using 10% acetic acid solution, stirred for 10 minutes and separated the methylene chloride layer. Aqueous layer was extracted with methylene chloride (100 ml), combined methylene chloride layer was dried over sodium sulphate (5 gm), distilled off methylene chloride completely under vacuum below 40° C. and stripped off methylene chloride with toluene (100 ml×2) to residue. The residue keto-montelukast was dissolved in toluene (40 ml) and used in part 2.

Part 2: THF (200 ml) and Cerium chloride (7.2 gm) was charged under nitrogen atmosphere, heated the contents to reflux and maintained for 3 hours. It was then, chilled to −5° C. and 3M methyl magnesium chloride (72 ml) was added dropwise over a period of 45 minutes and maintained at 0-5° C. for 2 hours. To this part 1 solution was added at −5 to 0° C. over a period of 1 hour and maintained for 1 hours. After completion of reaction, the mass was quenched into ice-water mixture (200 ml), pH of the reaction mass was adjusted to 6.0-6.5 using 10% acetic acid solution and extracted with ethyl acetate (100 ml×2). The ethyl acetate layer was dried over sodium sulphate (10 gm) and distilled off ethyl acetate completely under vacuum at less than 45° C. to residue. The residue was dissolved in ethyl acetate (80 ml), cyclohexylamine (2.9 gm) was added, stirred for 1 hour, cooled to 0-5° C. and maintained for 1 hour. The material was filtered, washed with chilled ethyl acetate (10 ml) and dried under vacuum at 40-45° C. to give Montelukast-cyclohexylamine salt (12.4 gm, 68% yield, 98.4% HPLC purity).

### Example 15

### Preparation of Montelukast Salts

Keto-montelukast-isopropylamine salt and Keto-montelukast-cyclohexylamine salt can be converted to Montelukast-isopropylamine salt, Montelukast-morpholine salt and Montelukast-cyclohexylamine salt by following the process as described in Example 14.

### Example 16

### Preparation of Montelukast Sodium

Montelukast-Isopropylamine salt (10 gm), methylene chloride (100 ml) and water (100 ml) was charged under nitrogen atmosphere and stirred for 10 minutes. The pH of the reaction mass was adjusted to 4.0-4.5 with 10% acetic acid solution, separated methylene chloride layer and extracted the aqueous layer with methylene chloride (50 ml), the combined methylene chloride layer was dried over sodium sulphate (5 gm) and distilled off methylene chloride completely under vacuum at 25-30° C. to residue. The residue was dissolved in methanol (60 ml), a solution of sodium hydroxide in methanol (0.68 gm in 30 ml of methanol) was added and stirred for 1 hour at 25-30° C., clarified, distilled off methanol completely under vacuum at less than 35° C., stripped off methanol with ethyl acetate (50 ml×3) to residue, the residue was dissolved in toluene (30 ml), n-heptane (100 ml) was added dropwise under nitrogen atmosphere at 25-30° C. and stirred for 30 minutes, filtered the material so obtained was dried under vacuum at 70-80° C. for 24 hours to give Montelukast sodium (8.5 gm, 90% yield, 99% HPLC purity).

### Example 17

### Preparation of Montelukast Sodium

Montelukast-morpholine salt and Montelukast-cyclohexylamine salt can be converted to Montelukast sodium by adopting the process as described in Example 16.

It will be appreciated that the invention may be modified within the scope of the claims.

1. A process for preparing a compound of formula 2: 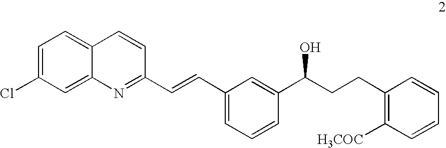
comprising reacting a compound of formula 1: 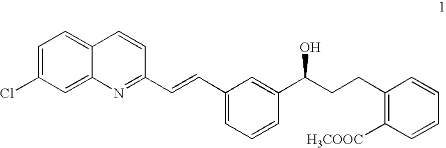
with lithium hexamethyldisilazide (LiHMDS) or N,O-dimethylhydroxylamine hydrochloride followed by reaction with methyl magnesium halide (MeMgX); preferably methyl magnesium bromide or methyl magnesium chloride.

2. (canceled)

3. The process according to claim 1, wherein the compound of formula 1 is prepared by reacting a compound of formula A: 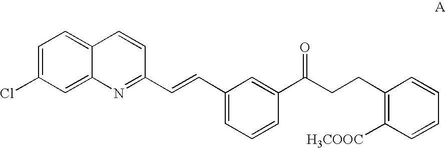
with a chiral reducing agent.

4. The process according to claim 3, wherein the chiral reducing agent is (−) diisopinocampheylchloroborane chloride; or (R)-borane dimethyl sulfide and a catalyst.

5. The process for preparing a compound of formula 3: 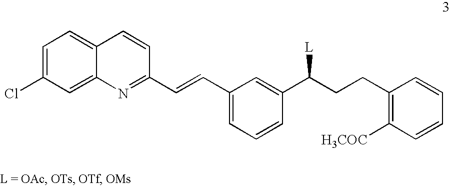
comprising, preparing compound of formula 2 according to claim 1 and reacting a compound of formula 2: 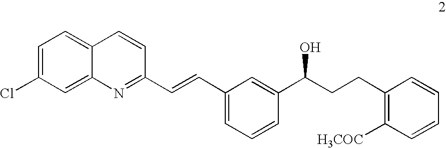
with an trifluoromethane sulfonic anhydride, p-toluene sulfonyl chloride, acetic anhydride or methane sulfonyl chloride.

6-9. (canceled)

10. The process for preparing a compound of formula 4, or a salt thereof: 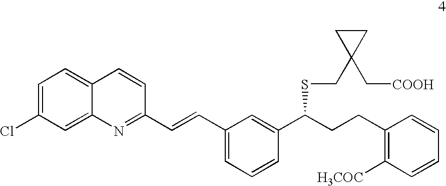
comprising, preparing compound of formula 3 according to claim 5 and reacting a compound of formula 3: 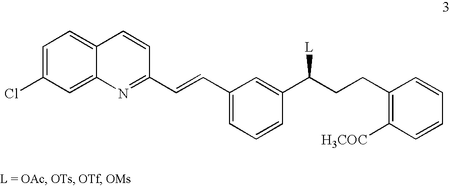
with 1-(mercaptomethyl)cyclopropane acetic acid in the presence of a base and an inert solvent.

11-13. (canceled)

14. A process for preparing a compound of formula 6: 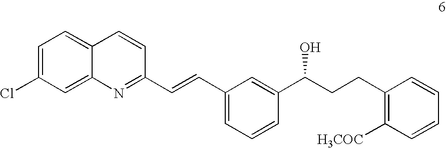
comprising reacting a compound of formula 5: 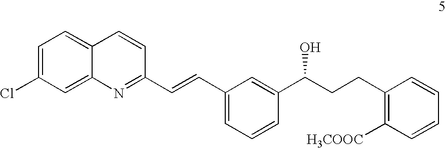
with lithium hexamethyldisilazide (LiHMDS) or N,O-dimethylhydroxylamine hydrochloride followed by reaction with methyl magnesium halide (MeMgX); preferably the methyl magnesium halide is methyl magnesium bromide or methyl magnesium chloride.

15-17. (canceled)

18. The process for preparing a compound of formula 7: 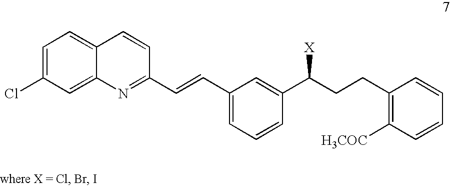
comprising, preparing compound 2 according to claim 1 and reacting a compound of formula 2 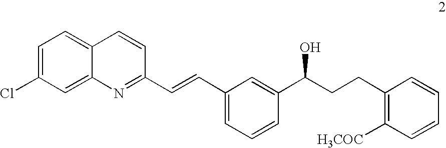
with a halogen donating compound in presence of a solvent.

19. (canceled)

20. The process for preparing a compound of formula 7: 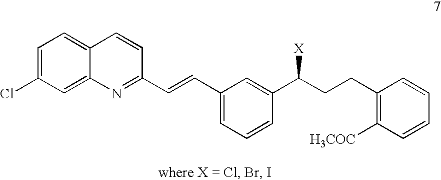
comprising, preparing compound 6 according to claim 14 and reacting a compound of formula 6: 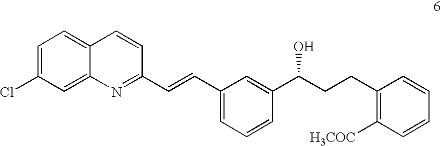
with a halogen donating compound in the absence of a solvent.

21-24. (canceled)

25. The process according to claim 18, wherein the hydroxyl group on compound (2) is converted to a trialkylsilyloxy group with a C1-C6 trialkylsilyl halide, preferably trimethylsilyl chloride to form a trialkylsilyloxy substituted compound.

26-28. (canceled)

29. The process according to claim 25, further comprising reacting the trialkylsilyloxy substituted compound with an alkali metal iodide or an ammonium iodide, preferably tetrabutyl ammonium iodide.

30-31. (canceled)

32. The process according to claim 20, wherein the compound is subjected to a Mitsunobu reaction with triphenylphosphine and diethyl azadicarboxylate, followed by reaction with an alkali metal iodide; preferably potassium iodide, cesium iodide or sodium iodide.

33. (canceled)

34. The process for preparing a compound of formula 4, or a salt thereof: 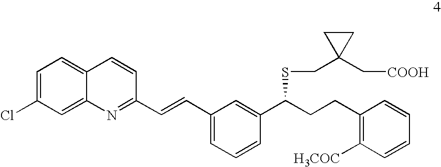
comprising, preparing compound 7 according to claim 18 and reacting a compound of formula 7: 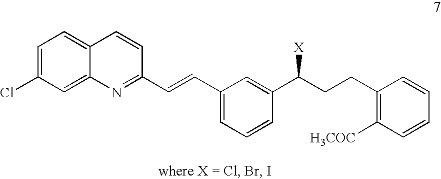
with 1-(mercaptomethyl)cyclopropane acetic acid in the presence of a base and an inert solvent.

35-37. (canceled)

38. The process for preparing a compound of formula I (montelukast), or a salt thereof: 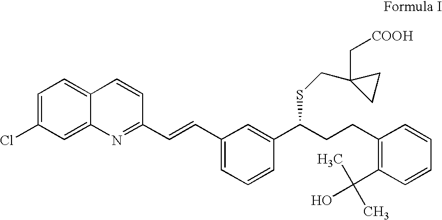
comprising, preparing compound 4 according to claim 10 and reacting a compound of formula 4, or a salt thereof: 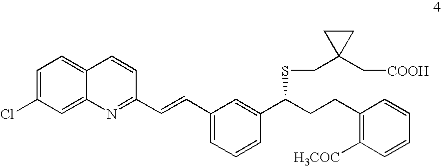
with methyl magnesium halide in the presence of an inert solvent.

39-43. (canceled)

44. A compound of formula 2:

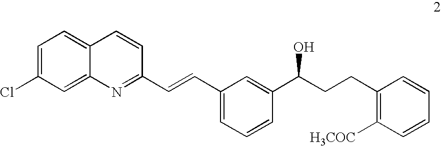

45. A compound of formula 3:

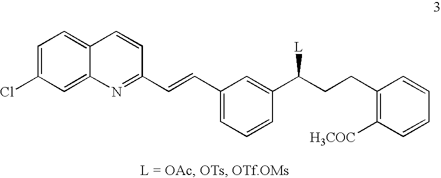

46. A compound of formula 4, or a salt thereof:

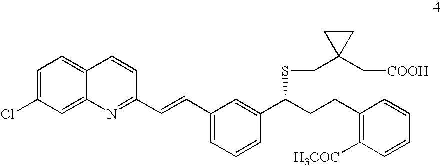

47. A compound of formula 6:

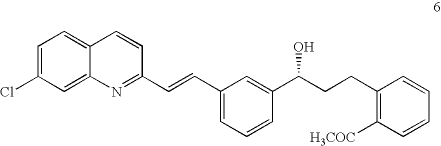

48. A compound of formula 7:

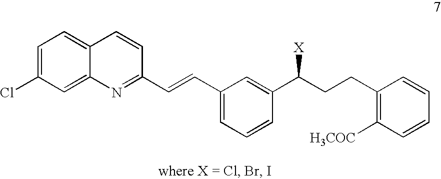

49-50. (canceled)

51. The process for preparing a compound of formula 4, or a salt thereof: 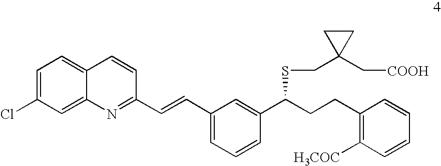
comprising, preparing compound 7 according to claim 20 and reacting a compound of formula 7: 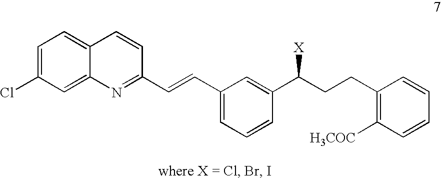
with 1-(mercaptomethyl)cyclopropane acetic acid in the presence of a base and an inert solvent.

52. The process for preparing a compound of formula I (montelukast), or a salt thereof: 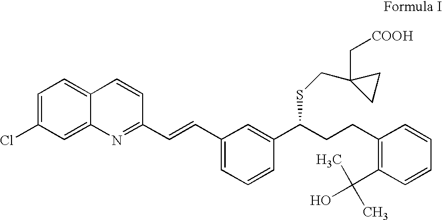
comprising, preparing compound 4 according to claim 34 and reacting a compound of formula 4, or a salt thereof: 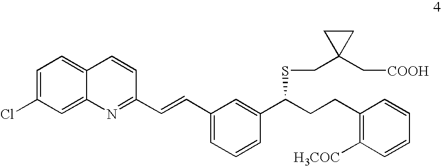
with methyl magnesium halide in the presence of an inert solvent.
